(19) United States
(12) Patent Application Publication
(10) Pub. No.: US 2020/0214791 A1
(43) Pub. Date: Jul. 9, 2020

(54) MEDICAL DEVICE RETENTION SYSTEM

(71) Applicant: OLYMPUS CORPORATION, Tokyo (JP)

(72) Inventor: Toru WADA, Niiza-shi (JP)

(73) Assignee: OLYMPUS CORPORATION, Tokyo (JP)

(21) Appl. No.: 16/819,323

(22) Filed: Mar. 16, 2020

Related U.S. Application Data
(63) Continuation of application No. PCT/JP2017/039550, filed on Nov. 1, 2017.

(51) Int. Cl. A61B 90/50; A61B 1/00; A61B 17/34

(52) U.S. Cl. CPC A61B 90/50; A61B 1/3132; A61B 17/3468; A61B 1/00147

(57) ABSTRACT

A medical device retention system has a tubular first retention portion into which a medical device is inserted and configured to hold the inserted medical device; a second retention portion having a spherical shape with a predetermined radius and having a central point at a distal end side of the supported first retention portion; and a clamp configured to clip the second retention portion, wherein the clamp switches the second retention portion between a first state and a second state when the second retention portion is clipped by the clamp, when the clamp switches the second retention portion into the first state, movement of the second retention portion is fixed, and when the clamp switches the second retention portion into the second state, the second retention portion is movable along a predetermined surface with the central point as a movement center.

This application is a continuation application of a PCT International Application No. PCT/JP2017/039550, filed on Nov. 1, 2017. The contents of the PCT International Application are incorporated herein by reference.

### BACKGROUND

### Field of the Invention

This invention relates to a medical device retention system configured to retain a medical device inserted into the body cavity of a patient such as a laparoscope, forceps, and the like.

### Description of Related Art

Conventionally, it is known that observation means such as the laparoscope and the treatment devices are inserted into the body of the patient to perform various treatment. It is possible that a surgeon handles a plurality of treatment devices at the same time while performing the treatment, and the treatment is complicated. Sometimes an assistance is assigned to stand immediately near the surgeon to retain the medical devices for the surgeon, however, it is possible to narrow the work space for the surgeon. Accordingly, there is a case in which a retention device configured to hold the medical device is provided instead of assigning the assistance. In the case of providing the retention device, when a position of the medical device retained by the retention device with respect to the treatment target is determined, or an orientation of the medical device is changed, it is necessary to avoid applying an excessive force to an insertion hole for the medical device that is formed on the abdominal wall and the like of the patient.

In Japanese Patent No. 3273084, a medical device holder apparatus is disclosed to hold the treatment devices and the endoscopes instead of the surgeon, wherein the treatment devices and the endoscopes are inserted into the abdominal cavity of the patient. This medical device holder apparatus is configured to have a link mechanism formed from a plurality of arms and the held medical devices move about a position near the insertion hole on the abdominal wall as a center such that it is possible to avoid the excessive force being applied to the insertion hole while determining the position or changing the orientation of the medical device.

In Japanese 2010-504990, an apparatus configured to automatically retain and guide the endoscope is disclosed. The apparatus is configured to automatically guide the held endoscope by an annular first guide portion provided in the horizontal direction and a circular second guide portion provided in the vertical direction according to an input from an arbitrary input means such as a key, a joy-stick and the like. Unintentional movement of the endoscope is avoided by automatizing the determination of the position of the held endoscope.

### SUMMARY

According to a first aspect of the present invention, A medical device retention system, has a tubular first retention portion into which a medical device is inserted, wherein the first retention portion is configured to hold the inserted medical device; a second retention portion configured to have a spherical shape with a predetermined radius, wherein the first retention portion is supported by the second retention portion, and the second retention portion has a central point at a distal end side of the supported first retention portion; and a clamp configured to clip the second retention portion. The clamp is configured to switch the second retention portion between a first state and a second state when the second retention portion is clipped by the clamp, wherein when the clamp switches the second retention portion into the first state, movement of the second retention portion is fixed, and when the clamp switches the second retention portion into the second state, the second retention portion is movable along a predetermined surface with the central point as a movement center.

According to a second aspect of the present invention, in the medical device retention system according to the first aspect, the clamp may have a clipping portion formed with a curvature same with a curvature of the second retention portion.

According to a third aspect of the present invention, in the medical device retention system according to the second aspect, the clamp may have an upper-side clip formed with the curvature same with the curvature of the second retention portion, and a lower-side clip formed with the curvature same with the curvature of the second retention portion.

According to a fourth aspect of the present invention, in the medical device retention system according to the third aspect, the clamp may be configured to switch the second retention portion between the first state and the second state by changing a distance between the upper-side clip and the lower-side clip.

According to a fifth aspect of the present invention, in the medical device retention system according to the fourth aspect, the upper-side clip may be formed from an elastically deformable material.

According to a sixth aspect of the present invention, the medical device retention system according to the fourth aspect may further have a biasing member configured to bias the upper-side clip so as to press the upper-side clip toward the second retention portion.

According to a seventh aspect of the present invention, the medical device retention system according to the sixth aspect may further have a connection member configured to connect the upper-side clip and the lower-side clip, wherein the biasing member is disposed in the connection member.

According to an eighth aspect of the present invention, the medical device retention system according to the third aspect may further have a connection member configured to connect the upper-side clip and the lower-side clip, wherein a space being surrounded by the upper-side clip, the lower-side clip, and the connection member may have a volume suitable for at least part of the second retention portion to enter.

According to a ninth aspect of the present invention, in the medical device retention system according to the first aspect, the second retention portion may be formed in a partial spherical shape.

According to a tenth aspect of the present invention, in the medical device retention system according to the seventh aspect, the second retention portion may be formed in a partial spherical shape smaller than a hemisphere surface.

According to an eleventh aspect of the present invention, in the medical device retention system according to the first aspect, the second retention portion may be formed from a transparent material.

According to a twelfth aspect of the present invention, in the medical device retention system according to the first aspect, the first retention portion may have a trocar configured to insert the medical device into a body cavity; and a trocar holder configured to hold the trocar on a wall of the body cavity.

According to a thirteenth aspect of the present invention, in the medical device retention system according to the first aspect, the first retention portion and the second retention portion may be formed integrally.

According to a fourteenth aspect of the present invention, in the medical device retention system according to the first aspect, the second retention portion may have a groove into which the first retention portion is inserted, and the first retention portion may be configured to be movable along the groove when the first retention portion is inserted into the groove.

According to a fifteenth aspect of the present invention, in the medical device retention system according to the first aspect, a convex projecting from the second retention portion may be disposed in a vicinity of an end of the second retention portion.

According to a sixteenth aspect of the present invention, in the medical device retention system according to the first aspect, the first retention portion may be configured to be supported by the second retention portion and movable along a virtual spherical surface while holding the medical device, and the virtual spherical surface may be defined by using the central point of the second retention portion as a center and a predetermined radius.

According to a seventeenth aspect of the present invention, in the medical device retention system according to the first aspect, the clamp may have a guide configured to guide the movement of the second retention portion.

According to an eighteenth aspect of the present invention, in the medical device retention system according to the first aspect, the clamp may have a brake configured to fix the movement of the second retention portion or release fixation of the movement of the second retention portion, and a brake operation portion configured to operate the brake portion may be disposed in the vicinity of the first retention portion.

According to a nineteenth aspect of the present invention, in the medical device retention system according to the eighteenth aspect, the brake operation portion may be attachable to and detachable from the medical device that is inserted into the first retention portion.

### BRIEF DESCRIPTION OF DRAWING

FIG. 1 is a schematic view showing an overall configuration of a medical device retention system according to a first embodiment of the present invention.

FIG. 2A is a schematic view showing a second retention portion of the medical device retention system.

FIG. 2B is a schematic view showing a support portion of the medical device retention system.

FIG. 3 is a schematic view showing the configuration of the medical device retention system.

FIG. 4 is a schematic view showing a configuration of a medical device retention system according to a second embodiment of the present invention.

FIG. 5 is a schematic view showing a configuration of a medical device retention system according to a third embodiment of the present invention.

FIG. 6A is a schematic view showing a configuration of a medical device retention system according to a fourth embodiment of the present invention.

FIG. 6B is a schematic view showing an operation of the medical device retention system.

FIG. 7 is a schematic view showing a configuration of a medical device retention system according to a fifth embodiment of the present invention.

FIG. 8 is a schematic view showing a modification example of the medical device retention system.

FIG. 9 is a schematic view showing a configuration of a medical device retention system according to a sixth embodiment of the present invention.

FIG. 10 is a schematic view showing a configuration of a medical device retention system according to a seventh embodiment of the present invention.

FIG. 11 is a schematic view showing a configuration of a medical device retention system according to an eighth embodiment of the present invention.

FIG. 12A is a schematic view showing a configuration of a second retention portion of a medical device retention system according to a ninth embodiment of the present invention.

FIG. 12B is a schematic view showing a configuration of a first retention portion of the medical device retention system.

FIG. 12C is a schematic view showing the configuration of the first retention portion.

FIG. 12D is a schematic view showing the configuration of the first retention portion.

FIG. 13A is a schematic view showing a relationship between the first retention portion and the second retention portion.

FIG. 13B is a schematic view showing the relationship between the first retention portion and the second retention portion.

### DETAILED DESCRIPTION OF EMBODIMENTS

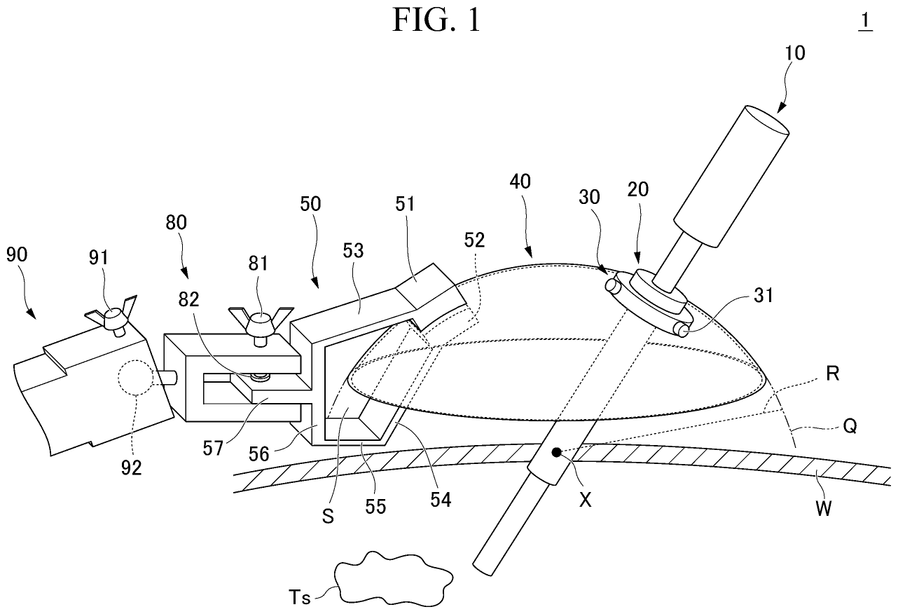

A first embodiment of the present invention will be described referring to enclosed FIG. 1 to FIG. 3. FIG. 1 is a schematic view showing an overall configuration of a medical device retention system 1 according to a first embodiment of the present invention. The medical device retention system 1 is a system configured to retain medical devices 10 inserted into the body of the patient such as a laparoscope, forceps and the like. The medical device retention system 1 has a first retention portion 20 configured to retain the medical devices 10, a second retention portion 40 configured to support the first retention portion 20, a clamp portion (clamp) 50 configured to clamp the second retention portion 40, a clamp fixing portion 80 configured to fix the clamp portion 50, and a fixing arm 90 connected to the clamp fixing portion 80.

The first retention portion 20 is formed in a tubular shape, and first retention portion 20 is configured to retain the medical device 10 inserted into the tubular shape thereof. For example, the first retention portion 20 may be a conventional trocar attached to an insertion hole formed on the abdominal wall W of the patient.

The second retention portion 40 has a center point X, and the second retention portion 40 is formed to have a partial spherical shape with a predetermined radius R. The second retention portion 40 is configured to support the first retention portion 20, and the second retention portion 40 has the center point X at a distal end side of the supported first retention portion 20. As shown in FIG. 1, the second retention portion 40 is configured to support the first retention portion (trocar) 20 by a support portion (support) 30. The position of the center point X of the second retention portion 40 is substantially coincided with the insertion hole on the abdominal wall W when the medical device retention system 1 is used.

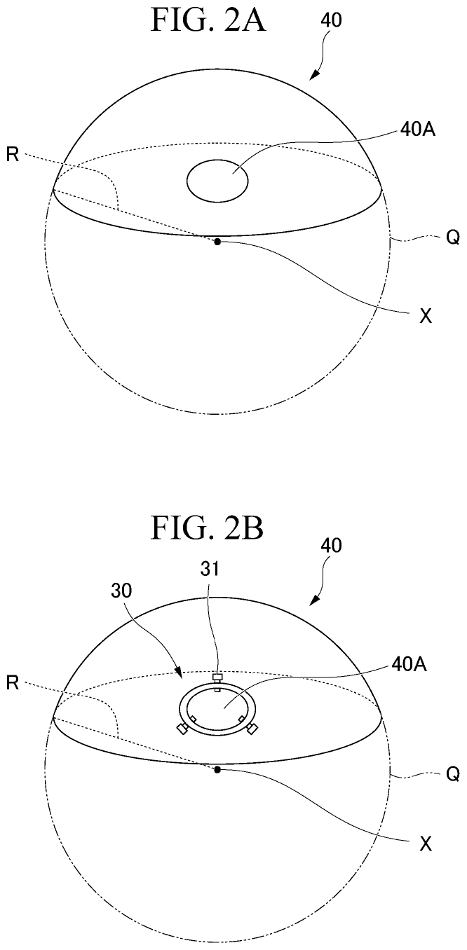

FIG. 2A is a schematic view showing a configuration of the second retention portion 40. As shown in FIG. 2A, the second retention portion 40 has a center point X, and the second retention portion 40 is formed to have the partial spherical shape with a predetermined radius R. The second retention portion 40 may be formed in a semi-spherical shape. However, as described below, since the second retention portion 40 moves in a predetermined range, it is preferable that the second retention portion 40 is formed in the partial spherical shape smaller than the semi-spherical shape.

A hole 40A is formed in the second retention portion 40 for the first retention portion (trocar) 20 to be inserted through. The second retention portion 40 is formed from the conventional metal material, resin material, and the like. It is preferable to use a transparent material to form the second retention portion 40 so as to make the inside of the second retention portion 40 easy to be visually observed.

FIG. 2B is a schematic view showing a configuration of the support portion 30. The support portion 30 is formed in a circular ring shape to surround a circumference of the hole 40A of the second retention portion 40. An inner circumferential surface of the support portion 30 substantially coincides with an opening end of the hole 40A. Within the support portion 30, a penetration hole is formed to penetrate the inner circumferential surface and the outer circumferential surface thereof. By inserting a screw member 31 into the penetration hole and fastening the screw member 31 therein, the support portion 30 is configured to support the first retention portion (trocar) 20 that is inserted through the hole 40A. In FIG. 2B, it is shown that three penetration holes and three screw members 31 are provided therein, however, the number of the penetration holes and the screw members may be single or multiple.

The support portion 30 is formed from a conventional metal material, resin material and the like. Attachment of the support portion 30 with respect to the second retention portion 40 can be performed by conventional methods such as an adhesion method and the like.

A clamp portion (clamp) 50 is configured to clamp the second retention portion 40. More specifically, the clamp portion 50 is configured to clamp the second retention portion 40 such that the second retention portion 40 can move around a central potion X as a center.

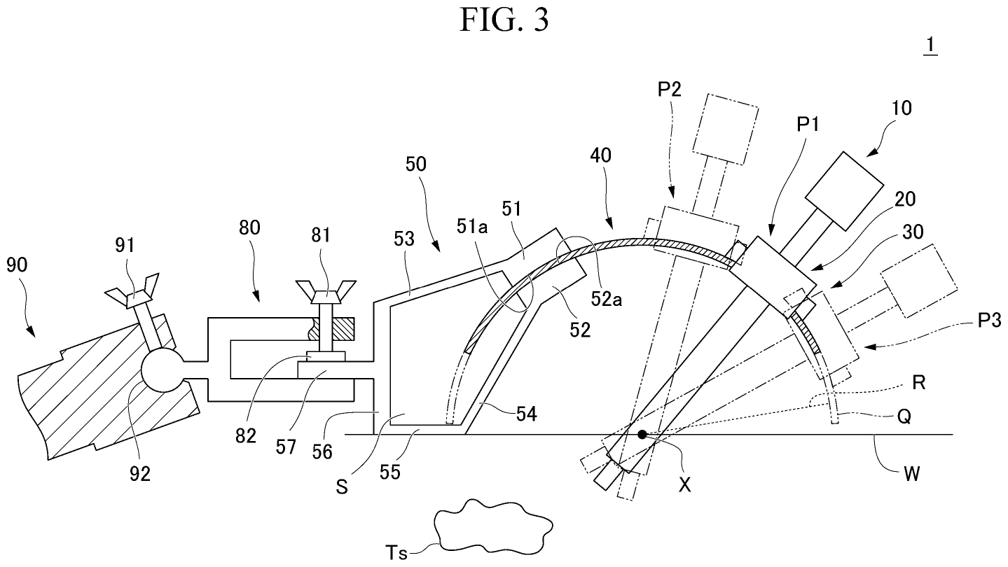

FIG. 3 is a schematic view showing a configuration of the medical device retention system 1. As shown in FIGS. 1 and 3, the clamp portion 50 has an upper-side clipping portion (upper-side clip) 51, a lower-side clipping portion (lower-side clip) 52, an upper-side connection portion (upper-side connector) 53, a lower-side connection portion (lower-side connector) 54, a middle connection portion (middle connector) 55, an upper-and-lower connection portion (upper-and-lower connector) 56, and a fastened portion (fastener) 57.

The upper-side clipping portion 51 is configured to come in contact with the outer circumferential surface of the second retention portion 40 when the upper-side clipping portion 51 clips the second retention portion 40. As shown in FIG. 3, a surface 51a of the upper-side clipping portion 51 at the side in contact with the second retention portion 40 is formed to be along the outer circumferential surface of the second retention portion 40. In other words, the surface 51a of the upper-side clipping portion 51 is formed to be concaved with a curvature same with the curvature of the second retention portion 40. The upper-side clipping portion 51 is connected with the upper end portion of the upper-and-lower connection portion 56 via the upper-side connection portion 53.

The lower-side clipping portion 52 is configured to come in contact with the inner circumferential surface of the second retention portion 40 when the lower-side clipping portion 52 clips the second retention portion 40. As shown in FIG. 3, a surface 52a of the lower-side clipping portion 52 at the side in contact with the second retention portion 40 is formed to be along the inner circumferential surface of the second retention portion 40. In other words, the surface 52a of the lower-side clipping portion 52 is formed to be convex with a curvature same with the curvature of the second retention portion 40. The lower-side clipping portion 52 is connected with the lower end portion of the upper-and-lower connection portion 56 via the lower-side connection portion 54 and the middle connection portion 55.

The clamp portion 50 is configured to clip the second retention portion 40 with a predetermined pressure by the upper-side clipping portion 51 and the lower-side clipping portion 52. By adjusting the predetermined pressure, the clamp portion 50 is configured to be able to fix the second retention portion 40, or move the second retention portion 40 so as to slide along the surface 51a and the surface 52a. In other words, the clamp portion 50 is configured to clamp the second retention portion 40 so as to be able to switch between a state (fixing state) in which the second retention portion 40 is fixed and a state (movement state) in which the second retention portion 40 is movable.

The pressure applied by the upper-side clipping portion 51 and the lower-side clipping portion 52 with respect to the second retention portion 40 is adjustable by changing a distance between the upper-side clipping portion 51 and the lower-side clipping portion 52. For example, the distance between the upper-side clipping portion 51 and the lower-side clipping portion 52 can be changed by using an elastically deformable member to form the upper-side connection portion 53 and making the upper-side connection portion 53 to deform elastically.

The fastened portion 57 is connected to the upper-and-lower connection portion 56 configured to connect the upper-side clipping portion 51 and the lower-side clipping portion 52. The fastened portion 57 is held and fixed by the clamp fixing portion 80.

As shown in FIGS. 1 and 3, the clamp portion 50 has a space S surrounded by the upper-side clipping portion 51, the lower-side clipping portion 52, the upper-side connection portion 53, the lower-side connection portion 54, the middle connection portion 55, and the upper-and-lower connection portion 56. The space S has a width such that part of the second retention portion 40 can enter the space S as shown below.

The upper-side clipping portion 51, the lower-side clipping portion 52, the upper-side connection portion 53, the lower-side connection portion 54, the middle connection portion 55, the upper-and-lower connection portion 56, and the fastened portion 57 are formed by conventional metal material or resin material and the like. Each portion can be individually formed, or integrally formed.

The clamp portion 50 only has to be configured to have the space S suitable for part of the second retention portion 40 to enter and clamp the second retention portion 40 such that the second retention portion 40 is movable around the central point X as the center, and the configuration of the clamp portion 50 is not limited to the configuration disclosed in FIGS. 1 and 3.

The clamp fixing portion 80 is configured to fix the clamp portion 50. The clamp fixing portion 80 is configured by a conventional fixing means. For example, as shown in FIGS. 1 and 3, the clamp fixing portion 80 is configured to fix the fastened portion 57 of the clamp portion 50 by operating the clamping bolt 81 so as to have a clamping plate 82 to descend.

Also, the clamp fixing portion 80 can be omitted by directly connecting the fastened portion 57 of the clamp portion 50 to the fixing arm 90.

The fixing arm 90 is configured to fix the clamp fixing portion 80. The fixing arm 90 is configured by conventional fixing means. For example, as shown in FIGS. 1 and 3, the fixing arm 90 has a clamping bolt 91 configured to fix the clamp fixing portion 80 and release the fixation of the clamp fixing portion 80, and a ball joint mechanism 92.

An operation example when the medical device retention system 1 having the above-described configuration is used will be described.

A user (operator) opens an insertion hole on the abdominal wall W while taking the position of the target portion Ts into consideration. Subsequently, the user inserts the first retention portion (trocar) 20 into the insertion hole and indwell the first retention portion 20 to the abdominal wall W.

Subsequently, the user inserts the first retention portion (trocar) 20 indwelled to the abdominal wall W through the hole 40A formed in the second retention portion 40. The first retention portion (trocar) 20 is supported by the second retention portion 40 by inserting the screw member 31 through the penetration hole formed on the lateral surface of the support portion 30 and fastening the screw member 31.

Subsequently, the user holds the second retention portion 40 supported by the first retention portion (trocar) 20 by the clamp portion 50. The user holds the second retention portion 40 by the clamp portion 50 such that the central point X of the second retention portion 40 is substantially same with the position of the insertion hole on the abdominal wall W. In this step, the second retention portion 40 is fixed by the clamp portion 50 (in the fixing state). Thereafter, the fastened portion 57 of the clamp portion 50 is fixed by the clamping bolt 81 and the clamping plate 82 of the clamp fixing portion 80. Also, the clamp fixing portion 80 is fixed by the clamping bolt 91 of the fixing arm 90.

According to the procedures shown above, the first retention portion (trocar) 20 may be inserted into the insertion hole on the abdominal wall W after being supported by the second retention portion 40.

Also, the first retention portion (trocar) 20 may be inserted through the hole 40A of the second retention portion 40 after the second retention portion 40 is clamped by the clamp portion 50.

Subsequently, the user inserts the medical device 10 such as the laparoscope and the like through the first retention portion (trocar) 20 and introduces the medical device 10 into the body of the patient.

According to the procedures so far, an environment is established such that the medical devices 10 such as the laparoscope, the forceps and the like are operable inside the body cavity while being held by the medical device retention system 1.

Subsequently, the user beings the treatment by the medical device 10. The user determines the position of the medical device 10 with respect to the target portion Ts and changes the orientation thereof while performing observation or treatment by the medical device 10.

In this situation, the user changes the distance between the upper-side clipping portion 51 and the lower-side clipping portion 52 of the clamp portion 50 to switch the second retention portion 40 from the fixing state to the movement state. For example, the user make the upper-side clipping portion 51 to be elastically deformed to raise the surface 51a of the upper-side clipping portion 51. Accordingly, the second retention portion 40 is switched from the fixing state to the movement state such that the second retention portion 40 is movable along the surface 52a of the lower-side clipping portion 52 and with the central point X as a movement center. The surface 52a of the lower-side clipping portion 52 has a curvature same with the curvature of the second retention portion 40. Accordingly, the surface 52a of the lower-side clipping portion 52 can guide (restrict) the second retention portion 40 so as to make the second retention portion 40 to move around the central point X as the movement center.

The first retention portion (trocar) 20 is supported by the second retention portion 40. Accordingly, in the situation when the second retention portion 40 is in the movement state, the first retention portion (trocar) 20 is movable together with the second retention portion 40. More specifically, the first retention portion (trocar) 20 is movable along a virtual spherical surface Q defined by the central point X of the second retention portion 40 and a predetermined radius R. Same with the second retention portion 40, the movement of the first retention portion (trocar) 20 is restricted by the movement along the virtual spherical surface Q, and the position of the first retention portion (trocar) 20 on the abdominal wall W does not change.

The medical device 10 is held by the first retention portion (trocar) 20. Accordingly, the user can determine the position and change the orientation of the medical device 10 by moving the medical device 10 together with the first retention portion (trocar) 20 along the virtual spherical surface Q.

In a situation when the user would like make the medical device 10 to stand in a direction closer to the vertical direction, the user intuitively operates the medical device 10 to make the medical device 10 to stand in the vertical direction. Following the operation by the user, as shown in FIG. 3, the first retention portion (trocar) 20 moves from a position P1 to a position P2 on the virtual spherical surface Q. In a situation when the user would like make the medical device to lie in a direction closer to the horizontal direction, the user intuitively operates the medical device 10 to make the medical device 10 to lie in the horizontal direction. Following the operation by the user, as shown in FIG. 3, the first retention portion (trocar) 20 moves from the position P1 to a position P3 on the virtual spherical surface Q.

A sequence of movements of the medical device 10 and the first retention portion (trocar) 20 occur around the central point X as the movement center, wherein the central point X is formed in the vicinity of the insertion hole on the abdominal wall W such that it is possible to prevent applying an excessive force with respect to the insertion hole on the abdominal wall W. In other words, the user can intuitively change the orientation of the medical device 10 and the like without applying the excessive force with respect to the abdominal wall W.

The clamp portion 50 is configured to have the space S into which part of the second retention portion 40 can enter. Accordingly, when the first retention portion (trocar) 20 moves along the spherical surface Q, the clamp portion 50 does not interfere with the second retention portion 40.

Once the user finishes the position determination and orientation change of the medical device 10, the user terminates the raise of the upper-side clipping portion 51 toward the upper side of the surface 51a of the upper-side clipping portion 51. The clamp portion 50 clamps the second retention portion 40 by the upper-side clipping portion 51 and the lower-side clipping portion 52 again. Thus, the second retention portion 40 enters the fixing state from the movement state. Similarly, the movement of the first retention portion (trocar) 20 supported by the second retention portion 40 along the virtual spherical surface Q is fixed. Accordingly, the first retention portion (trocar) 20 is configured to hold the medical device 10 so as to maintain the position of the medical device 10 that the position determination and the orientation change are finished.

As described above, according to the medical device retention system 1 according to the present embodiment, the user can determine the position and change the orientation of the medical device 10 by moving the first retention portion (trocar) 20 that is supported by the partial-spherical shaped second retention portion 40 along the virtual spherical surface Q that is defined by the central point X of the second retention portion 40 and the predetermined radius R. Accordingly, the user can intuitively change the orientation of the medical device 10 held by the first retention portion (trocar) 20 toward a desiring direction.

Also, the movement of the first retention portion (trocar) 20 holding the medical device 10 is restricted to be along the virtual spherical surface Q defined by the central point X of the partial-spherical shaped second retention portion 40 and the predetermined radius R. Accordingly, by substantially coinciding the central point X and the insertion hole on the abdominal wall W, there is no excessive force applied to the insertion hole on the abdominal wall W while changing the orientation of the medical device 10.

Also, the movement of the first retention portion (trocar) 20 along the virtual spherical surface Q can be controlled by the partial-spherical shaped second retention portion 40 and the clamp portion 50. Accordingly, it is possible to make the configuration of the medical device retention system 1 to be simple.

Furthermore, the first retention portion (trocar) 20, the second retention portion 40, and the clamp portion 50 are configured to be separate with each other. Accordingly, for example, the first retention portion (trocar) 20 and the second retention portion 40 can be configured as disposable members and the clamp portion 50 can be configured to be sterilized as a reused member. Accordingly, it is possible to configure the medical device retention system 1 to be superior in both of sanitation aspect and cost aspect.

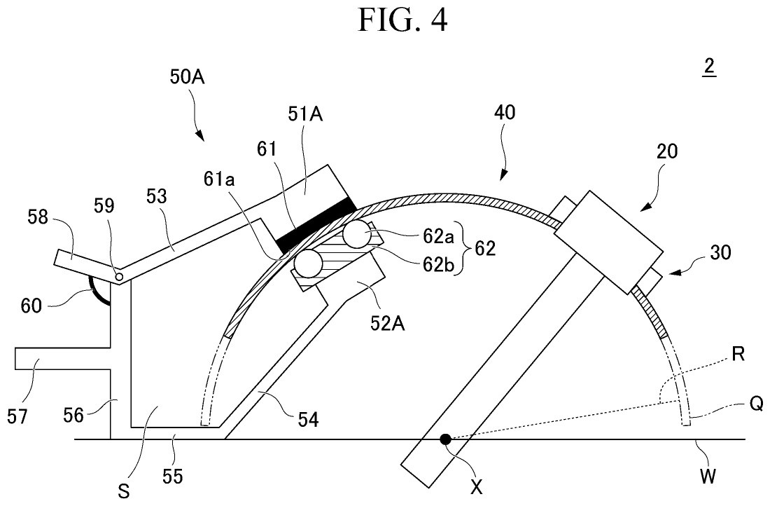

Next, a second embodiment of the present invention will be described by referring to FIG. 4. The present embodiment is different from the first embodiment in the configuration aspect of the clamp portion. In the description below, the described common configurations will be referred by the same reference signs and the duplicated description will be omitted.

FIG. 4 is a schematic view showing a medical device retention system 2 according to a second embodiment of the present invention. In order to make FIG. 4 easy to view, the medical device 10, the clamp fixing portion 80, and the fixing arm 90 are omitted.

The medical device retention system 2 has a clamp portion (clamp) 50A. The clamp portion 50A has an upper-side clipping portion 51A with a brake portion (brake) 61, a lower-side clipping portion 52A with a guide portion (guide) 62, and a brake-release lever (brake operation portion) 58.

As shown in FIG. 4, the upper-side connection portion 53 is rotatably connected to the upper end portion of the upper-and-lower connection portion 56 via a shaft member 59.

The clamp portion 50A has the brake-release lever 58 which is formed to extend from the position of the shaft member 59. A biasing member 60 is attached therebetween the brake-release lever 58 and the upper-and-lower connection portion 56. The biasing member 60 may be a conventional spring and the like, and the biasing member 60 is configured to bias the upper-side clipping portion 51A such that the upper-side clipping portion 51A presses the second retention portion 40 with a predetermined pressure.

The upper-side clipping portion 51A has a brake portion 61 at the side in contact with the second retention portion 40. As described above, the upper-side clipping portion 51A (brake portion 61) is biased by the biasing member 60 so as to press the second retention portion 40 with the predetermined pressure. A surface 61a of the brake portion 60 at the side in contact with the second retention portion 40 is formed to have a curvature same with the curvature of the second retention portion 40.

The lower-side clipping portion 52A has a guide portion 62 at the side in contact with the second retention portion 40. The guide portion 62 has a plurality of balls 62a and a ball support portion 62b. The plurality of balls 62a abut on the inner circumferential surface of the second retention portion 40. A number and arrangement of the plurality of balls 62a can be suitably determined. As shown in FIG. 4, the ball support portion 62b is configured to have a thickness at an outer edge portion thereof smaller than a thickness at a central portion thereof so as to avoid the interference with the movement of the second retention portion 40.

An operation example when the medical device retention system 2 having the above-described configuration is used will be described.

The clamp portion 50A is configured to clamp the second retention portion 40 with a predetermined pressure by the upper-side clipping portion 51A and the lower-side clipping portion 52A. More specifically, the second retention portion 40 is in the fixing state since the brake portion 61 biased by the biasing member 60 is configured to press the second retention portion 40 toward the lower-side clipping portion 52A (the plurality of balls 62a) with the predetermined pressure.

The user presses the brake-release lever 58 downwardly at the time of changing the orientation of the medical device 10. Due to this operation by the user, the upper-side connection portion 53 rotates around the shaft member 59 and the brake portion 61 departs from the outer circumferential surface of the second retention portion 40. A rotatable angle of the upper-side connection portion 53 at this time is determined such that a slight gap (distance) between the brake portion 61 and the outer circumferential surface of the second retention portion 40 is generated. Accordingly, the second retention portion 40 is switched from the fixing state to the movement state.

The lower-side clipping portion 52A has the guide portion 62 at the side in contact with the second retention portion 40. The plurality of balls 62a provided in the guide portion 62 abut on the inner circumferential surface of the second retention portion 40 such that the user can move the second retention portion 40 with the central point X as the movement center by the guidance of the guide portion 62. In other words, in the movement state of the second retention portion 40, the user can move the first retention portion (trocar) 20 along the virtual spherical surface Q by the guidance of the guide portion 62.

After the change of the orientation of the medical device 10 is finished, the user returns the brake-release lever 58 to the original position. Since the brake-release lever 58 is biased by the biasing member 60, once the user releases his/her hand from the brake-release lever 58, the brake-release lever 58 can return to the original position.

When the brake-release lever 58 returns to the original position, the brake portion 61 presses the second retention portion 49 with the predetermined pressure again. Thus, the second retention portion 40 is switched from the movement state to the fixing state.

In the medical device retention system 2 according to the present embodiment, similar to the first embodiment, the medical device retention system 2 has a simple configuration, and it is possible to intuitively change the orientation of the held medical device and the like.

According to the present embodiment, the clamp portion 50A is has the guide portion 62 configured to guide the movement of the second retention portion 40 such that it is possible to smoothly move the second retention portion 40.

Also, according to the present embodiment, the clamp portion 50A has the brake portion 61 configured to fix the movement of the second retention portion 40 and release the fixation of the movement of the second retention portion 40 such that there is no risk occurring since the fixation is forgotten and it is possible to definitely fix the movement of the second retention portion 40.

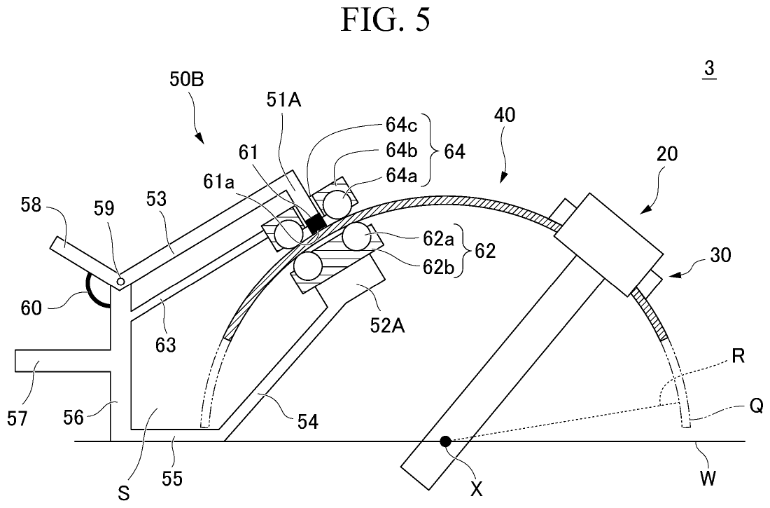

Next, a third embodiment of the present invention will be described by referring to FIG. 5. The present embodiment is different from the second embodiment in that a guide portion is disposed to abut on the outer circumferential surface of the second retention portion 40.

FIG. 5 is a schematic view showing a medical device retention system 3 according to a third embodiment of the present invention. In FIG. 5, in order to make the figure easy to view, the medical device 10, the clamp fixing portion 80, and the fixing arm 90 are omitted.

The medical device retention system 3 has a clamp portion (clamp) 50B. The clamp portion 50B has a guide portion 64 abutting on the outer circumferential surface of the second retention portion 40.

The clamp portion 50B has a connection portion 63 extending from a position being in the slightly downward vicinity of the upper end portion of the upper-and-lower connection portion 56. The connection portion 63 is configured to connect the guide portion 64 and the upper-and-lower connection portion 56.

The guide portion 64 has a plurality of balls 64a, a ball support portion 64b, and a penetration hole 64c. The plurality of balls 64a abut on the outer circumferential surface of the second retention portion 40. A number and the arrangement of the plurality of balls 64a can be suitably determined. As shown in FIG. 5, the ball support portion 64b is configured to have a thickness at the outer edge portion larger than a thickness at the central portion thereof so as to prevent the plurality of balls 64a from slipping. Also, the guide portion 64 has the penetration hole 64c so as to not to disturb the movement of the upper-side clipping portion 51A. The upper-side clipping portion 51A is configured to access the outer circumferential surface of the second retention portion 40 via the penetration hole 64c. A dimension of the upper-side clipping portion 51A can be suitable changed in consideration with the dimension of the penetration hole 64c.

In the medical device retention system 3 according to the present embodiment, similar to the second embodiment, the medical device retention system 3 has a simple configuration, and it is possible to intuitively change the orientation of the held medical device and the like.

According to the present embodiment, the clamp portion 50B has the guide portion 64 in contact with the outer circumferential surface of the second retention portion 40 in addition to the guide portion 62 in contact with the inner circumferential surface of the second retention portion 40. The user can guide the movement of the second retention portion 40 with the central point X as the movement center by using the guide portion 62 and the guide portion 64 as a guide. Accordingly, it is possible to more smoothly move the second retention portion 40.

Also, according to the present embodiment, the second retention portion 40 is always clamped by the guide portion 62 and the guide portion 64 with the predetermined pressure. Accordingly, in the movement state of the second retention portion 40, it is possible to reduce the cases in which the second retention portion 40 suddenly slips from the clamp portion 50B.

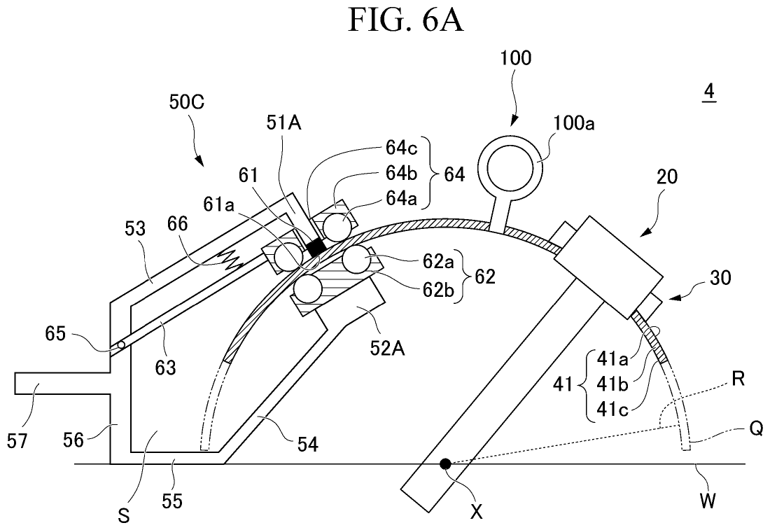

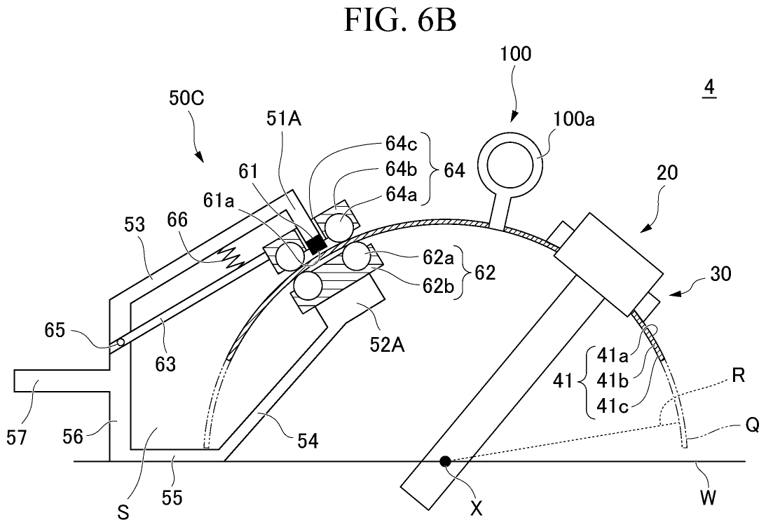

Next, a fourth embodiment of the present invention will be described by referring to FIGS. 6A-6B. The present embodiment is different from the third embodiment in that the brake operation portion is disposed near the first retention portion through which the medical device 10 is inserted.

FIG. 6A is a schematic view showing a medical device retention system 4 according to a fourth embodiment of the present invention. In FIG. 6A, in order to make the figure easy to view, the medical device 10, the clamp fixing portion 80, and the fixing arm 90 are omitted.

The medical device retention system 4 has a second retention portion 41, a clamp portion (clamp) 50C and a brake-release operation portion (brake operation portion, brake operation ring) 100.

Different from the third embodiment, the second retention portion 41 is formed from an outermost layer 41a, an intermediate layer 41b, and an innermost layer 41c.

Different from the third embodiment, the clamp portion 50C does not have the brake-release lever 58, the shaft member 59, and the biasing member 60. On the other hand, the clamp portion 50C has a shaft member 65 and a biasing member 66.

As shown in FIG. 6A, the connection portion 63 is rotatably connected with the upper-and-lower connection portion 56 via the shaft member 65. In the clamp portion 50C, the shaft member 59 of the clamp portion 50B according to the third embodiment is not provided such that the upper-side connection portion 53 is not rotatably connected to the upper end portion of the upper-and-lower connection portion 56.

The biasing member 66 is attached therebetween the connection portion 63 and the upper-side connection portion 53. The biasing member 66 is formed form the conventional spring and the like, and the biasing member 66 is configured to bias the guide portion 64 such that the guide portion 64 abuts on the outermost layer 41a of the second retention portion 41 with a predetermined pressure.

The second retention portion 41 is configured by the outermost layer 41a formed from a rigid material, the intermediate layer 41b formed from a soft material, and the innermost layer 41c formed from a rigid material. The outermost layer 41a configures the outer circumferential surface of the second retention portion 41 and the outermost layer 41a abuts on the brake portion 61 and the guide portion 64. The innermost layer 41c configures the inner circumferential surface of the second retention portion 41 and the innermost layer 41c abuts on the guide portion 62. The intermediate layer 41b is sandwiched between the outermost layer 41a and the innermost layer 41c.

As shown in FIG. 6A, the brake-release operation portion 100 is attached to the second retention portion 41. The lower end portion of the brake-release operation portion 100 is connected with a surface of the innermost layer 41c at the side of the intermediate layer 41b. In the upper end portion of the brake-release operation portion 100, a ring-shaped handle portion 100a is formed for the user to lay fingers and the like.

An operation example when the medical retention system 4 having the above-described configuration is used will be described by referring to FIGS. 6A and 6B. FIG. 6B is a schematic view showing the operation of the medical device retention system 4.

The clamp portion 50C has the upper-side clipping portion 51A (brake portion 61) and the lower-side clipping portion 52A (guide portion 62) clamping the second retention portion 41 with the predetermined pressure such that the second retention portion 41 is in the fixing state.

When the user performs the operation of changing the orientation of the medical device 10, the user lays the forefinger on the handle portion 100a of the brake-release operation portion 100. The user uses the thumb to press the outermost layer 41a of the second retention portion 41 while using the forefinger to raise the handle portion 100a. By the operations of the user, the outermost layer 41a of the second retention portion 41 is pressed toward the innermost layer 41c side and the innermost layer 41c is raised toward the outermost layer 41a side. The intermediate layer 41b of the second retention portion 41 is formed by the soft material such that the intermediate layer 41b deforms following the movement of the outermost layer 41a and the innermost layer 41c. That is, as shown in FIG. 6B, the intermediate layer 41b deforms such that the film thickness is decreased.

As shown in FIG. 6B, the film thickness of the intermediate layer 41b is decreased such that the outermost layer 41a of the second retention portion 41 departs from the brake portion 61 of the upper-side clipping portion 51A. The guide portion 64 continues to abut on the outermost layer 41a since the guide portion 64 is biased by the biasing member 66 so as to abut on the outermost layer 41a of the second retention portion 41. Accordingly, the second retention portion 41 switches from the fixing state to the movement state.

When the user finishes changing the orientation of the medical device 10, the user left the hand from the brake-release operation portion 100. The intermediate layer 41b of the second retention portion 41 restores to the original shape such that the outermost layer 41a of the second retention portion 41 abuts on the surface 61a of the brake portion 61 again. Accordingly, the second retention portion 41 switches from the movement state to the fixing state.

In the medical device retention system 4 according to the present embodiment, similar to the third embodiment, the medical device retention system 4 has a simple configuration, and it is possible to intuitively change the orientation of the held medical device and the like.

According to the present embodiment, the brake-release operation portion 100 is disposed in the vicinity of the first retention portion 20. The user can grasp the medical device inserting through the first retention portion 20 by one hand while operating the brake-release operation portion 100 by the other hand so as to fix the movement of the second retention portion 41 or release the fixation of the movement thereof. Accordingly, the operability of the medical device retention system 4 can be improved.

According to the present embodiment, the example in which the intermediate layer 41b of the second retention portion 41 is formed from the soft material and the intermediate layer 41b is formed all over between the outermost layer 41a andthe innermost layer 41c is described. However, instead of the intermediate layer 41b, a plurality of elastic members may be dotted therebetween so as to support and couple the outermost layer 41a and the innermost layer 41c. In this case, in the area where the plurality of elastic members are not provided, a gap between the outermost layer 41a and the innermost layer 41c is formed.

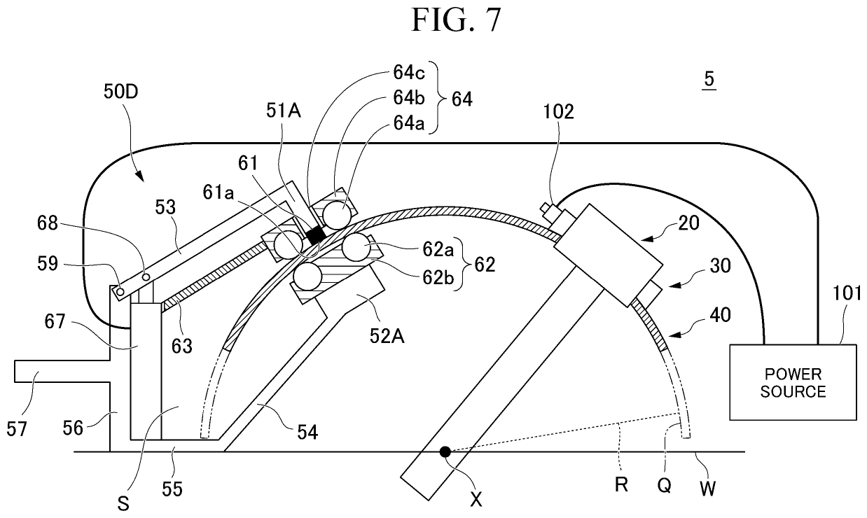

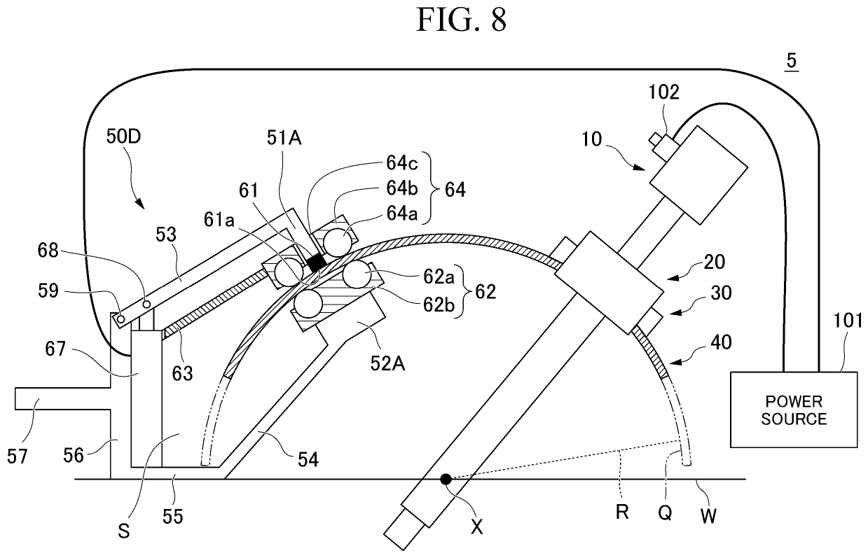

Next, a fifth embodiment of the present invention will be described by referring to FIGS. 7 and 8. The present embodiment is different from the third embodiment in that a brake operation portion is disposed near the first retention portion 20 through which the medical device 10 is inserted and operation of the brake operation portion is performed by a switch operation.

FIG. 7 is a schematic view showing a configuration of a medical device retention system 5 according to the fifth embodiment of the present invention. In FIG. 7, in order to make the figure easy to view, the medical device 10, the clamp fixing portion 80, and the fixing arm 90 is omitted.

The medical device retention system 5 has a clamp portion (clamp) 50D, a driver 67, a power supply portion 101, and a brake operation portion (brake operation button) 102. The clamp portion 50D is different from that according to the third embodiment in that the clamp portion 50D does not have the brake-release lever 58 and the biasing member 60. On the other hand, the clamp portion 50D has the driver 67 configured to drive the upper-side connection portion 53 so as to make the upper-side connection portion 53 to rotate around the shaft member 59.

As shown in FIG. 7, the upper end portion of the driver 67 is connected to the upper-side connection portion 53 via the shaft member 68. A penetration hole for the driver 67 to be inserted through is formed in the connection portion 63. The driver 67 is connected with the power supply portion 101. The power supply portion 101 is connected to the brake operation portion 102. The brake operation portion 102 is attached to the support portion 30 and the brake operation portion 102 is configured to be able to perform the switch operation.

The user performs the switch operation of the brake operation portion 102 to control ON/OFF of the driver 67 connected with the power supply portion 101.

For example, when the user presses the switch of the brake operation portion 102 to turn on the driver 67, the driver 67 is raised to make the upper-side connection portion 53 to rotate around the shaft member 59. Once the upper-side connection portion 53 is raised, the brake portion 61 departs from the outer circumferential surface of the second retention portion 40. Accordingly, the second retention portion 40 is switched from the fixing state to the movement state.

Again, when the user presses the switch of the brake operation portion 102 to turn off the driver 67, the operation of raising the upper-side connection portion 53 by the driver 67 is finished. When the upper-side connection portion 53 returns to the original position, the brake portion 61 presses the second retention portion 40 with the predetermined pressure again. Accordingly, the second retention portion 40 switches from the movement state to the fixing state.

For example, the driver 67 can be configured by the conventional motor, solenoid, and the like. In the case of using the solenoid to configure the driver 67, it is possible to operate the iron core as the magnetic body only by supplying current to the coil and make the configuration of the driver 67 to be simple; thus, this configuration is preferable.

In the medical device retention system 5 according to the present embodiment, similar to the third embodiment, the medical device retention system 4 has a simple configuration, and it is possible to intuitively change the orientation of the held medical device and the like.

According to the present embodiment, the brake operation portion 102 is disposed to be in the vicinity of the first retention portion 20 and has the configuration capable of performing the switch operation. Accordingly, it is possible to further improve the operability of the medical device retention system 5.

As a modification example of the present embodiment, the brake operation portion 120 may be attached to the medical device 10 inserting through the first retention portion 20 and the brake operation portion 120 may be attachable to and detachable from the medical device. FIG. 8 is a schematic view showing the modification example of the medical device retention system 5. In FIG. 8, in order to make the figure easy to view, the clamp fixing portion 80 and the fixing arm 90 are omitted. As shown in FIG. 8, the brake operation portion 102 may be attached to a position near the operation portion of the medical device 10 and the brake operation portion 102 may be attachable to and detachable from the medical device 10. Accordingly, the user can operate the brake operation portion 102 by the hand holding the medical device 10 so as to further improve the operability of the medical device retention system 5.

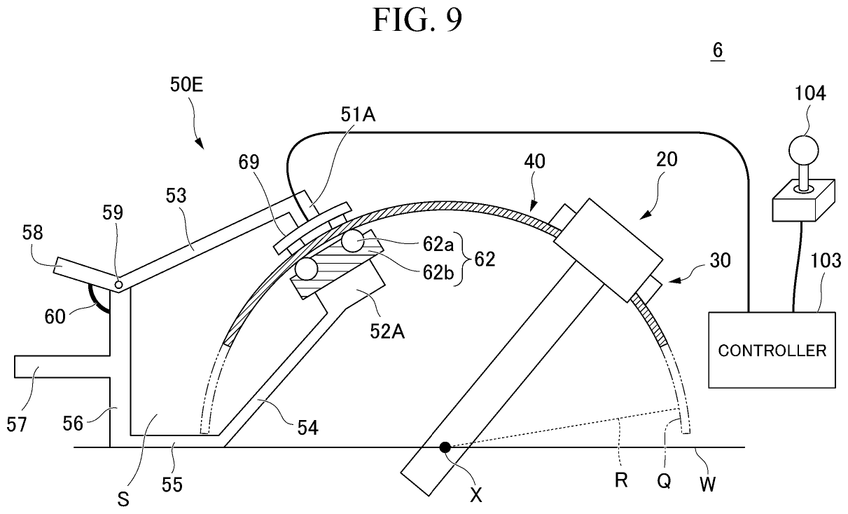

Next, a sixth embodiment of the present invention will be described by referring to FIG. 9. The present embodiment is different from the second embodiment in the configuration embodiment of the upper-side clipping portion.

FIG. 9 is a schematic view showing a configuration of a medical device retention system 6 according to the sixth embodiment of the present invention. In FIG. 9, in order to make the figure easy to view, the medical device 10, the clamp fixing portion 80, and the fixing arm 90 are omitted.

The medical device retention system 6 has a clamp portion (clamp) 50E, a control portion 103, and a holder-orientation operation portion (brake operation portion) 104. The clamp portion 50E is different from that according to the second embodiment, and the clamp portion 50E has an actuator portion 69 disposed at the side of the upper-side clipping portion 51A in contact with the second retention portion 40.

As shown in FIG. 9, the actuator portion 69 is connected to the control portion 103 configured by a Central Processing Unit (CPU) and the like. The actuator portion 69 has an actuator configured to drive the second retention portion 40 in accordance with the control signal transmitted from the control portion 103. The control portion 103 is connected to the holder-orientation operation portion 104 which is configured by a joystick and the like.

The clamp portion 50E has the upper-side clipping portion 51A and the lower-side clipping portion 52A configured to clamp the second retention portion 40 with a predetermined pressure. More specifically, the actuator portion 69 being biased by the biasing member 60 presses the second retention portion 40 toward the lower-side clipping portion 52A (ball 62a) with the predetermined pressure such that the second retention portion 40 is in the fixing state. In other words, the actuator portion 69 plays a role as a brake portion in the clamp portion 50E.

The control portion 103 is configured to generate the control signal in accordance with the joystick operation of the holder orientation operation portion 104 by the user, and transmits the generated control signal to the actuator portion 69. When the actuator portion 69 receives the control signal, the actuator portion 69 makes the second retention portion 40 to move (movement state) so as to correspond to the joystick operations of the holder orientation operation portion 104 by the user.

When the joystick operation of the holder orientation operation portion 104 by the user is finished, the actuator portion 69 is configured to press the second retention portion 40 toward the lower-side clipping portion 52A with the predetermined pressure such that the second retention portion 40 switches to the fixing state again.

In the medical device retention system 6 according to the present embodiment, similar to the second embodiment, the medical device retention system 4 has a simple configuration, and it is possible to intuitively change the orientation of the held medical device and the like.

According to the present embodiment, the upper-side clipping portion 51A has the actuator portion 69 and the actuator portion 69 is connected to the holder orientation operation portion 104 via the control portion 103. Accordingly, the user can easily and accurately move the second retention portion 40 by the operation of the holder orientation operation portion 104.

Also, the medical device retention system 6 according to the present embodiment also has the manual brake-release lever 58 in the clamp portion 50E besides the holder orientation operation portion 104 capable of performing joystick operation. The user can operate the holder orientation operation portion 104 to electrically move the second retention portion 40, and the user can operate the brake-release lever 58 to manually move the second retention portion 40. Accordingly, the user can select the most suitable movement means for the second retention portion 40 in accordance with the various situations during the treatment.

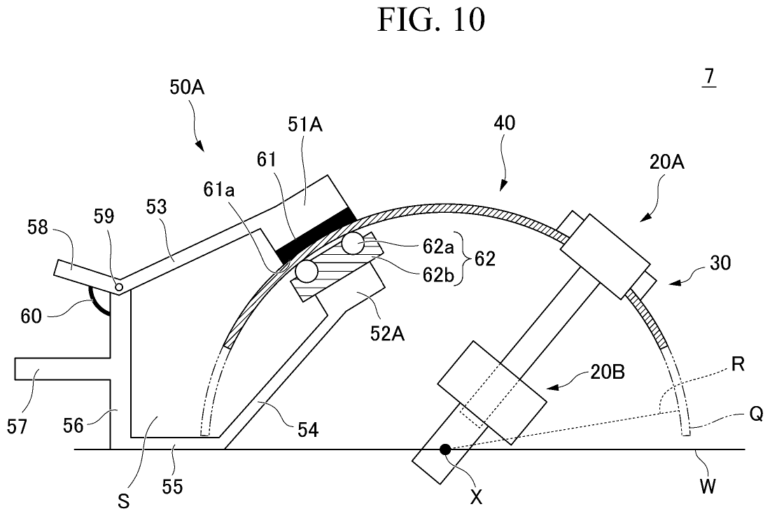

Next, a seventh embodiment of the present invention will be described by referring to FIG. 10. The present embodiment is different from the second embodiment in the configuration aspect of the first retention portion.

FIG. 10 is a schematic view showing a configuration of a medical device retention system 7 according to the seventh embodiment of the present invention. In FIG. 10, in order to make the figure easy to view, the medical device 10, the clamp fixing portion 80, and the fixing arm 90 are omitted.

The medical device retention system 7 has a first retention portion 20A having a distal end to which a trocar 20B is attachable. As shown in FIG. 10, the first retention portion 20A is configured as a trocar adapter configured to hold the trocar 20B, wherein the trocar 20B is attached to the distal end of the first retention portion 20A. The support from the second retention portion 40 with respect to the first retention portion (trocar adapter) 20A is performed by the support portion 30. The medical device 10 is inserted into the body of the patient via the first retention portion (trocar adapter) 20A and the trocar 20B.

According to the present embodiment, the second retention portion 40 may be formed from a transparent material. The second retention portion 40 may have a plurality of small holes as meshes of a net. Accordingly, it is easy for the user to confirm the positions of the first retention portion (trocar adapter) 20A and the trocar 20B when the user attaches the trocar 20B to the first retention portion (trocar adapter) 20A. Also, in the situation when the second retention portion 40 has the plurality of holes, a friction between the second retention portion 40 and the brake portion 61 becomes large such that the second retention portion 40 can be stably fixed.

In the medical device retention system 7 according to the present embodiment, similar to the second embodiment, the medical device retention system 4 has a simple configuration, and it is possible to intuitively change the orientation of the held medical device and the like.

According to the present embodiment, the first retention portion 20A is configured as the trocar adapter which can hold the trocar 20B and the first retention portion 20A can compensate the length of the trocar 20B. Accordingly, even if the trocar 20B is short, the distance between the second retention portion 40 and the abdominal wall W can be kept and it is possible to move the medical device 10 along the virtual spherical surface Q.

Next, an eighth embodiment of the present invention will be described by referring to FIG. 11. The present embodiment is different from the seventh embodiment in the configuration aspect of the first retention portion and the second retention portion.

FIG. 11 is a schematic view showing a configuration of a medical device retention system 8 according to an eighth embodiment of the present invention. In FIG. 11, in order to make the figure easy to view, the medical device 10, the clamp fixing portion 80, and the fixing arm 90 are omitted.

The medical device retention system 8 has a first retention portion (trocar adapter) 20C and a second retention portion 42. As shown in FIG. 11, the first retention portion (trocar adapter) 20C is formed integrally with the second retention portion 42. For example, the first retention portion (trocar adapter) 20C and the second retention portion 42 are formed from the conventional resin material and the like.

In the medical device retention system 8 according to the present embodiment, similar to the seventh embodiment, the medical device retention system 4 has a simple configuration, and it is possible to intuitively change the orientation of the held medical device and the like. Also, even in the case in which the trocar 20B is short, it is possible to keep the distance between the second retention portion 42 and the abdominal wall W, and it is possible to move the medical device 10 along the virtual spherical surface Q.

According to the present embodiment, the first retention portion (trocar adapter) 20C is formed integrally with the second retention portion 42. Accordingly, the support portion configured to attach the first retention portion (trocar adapter) 20C to the second retention portion 42 is unnecessary such that it is possible to reduce the number of the components to configure the medical device retention system 8. Also, the procedures for attaching the first retention portion (trocar adapter) 20C to the second retention portion 42 are also unnecessary such that the setting procedures of the medical device retention system 8 become easy.

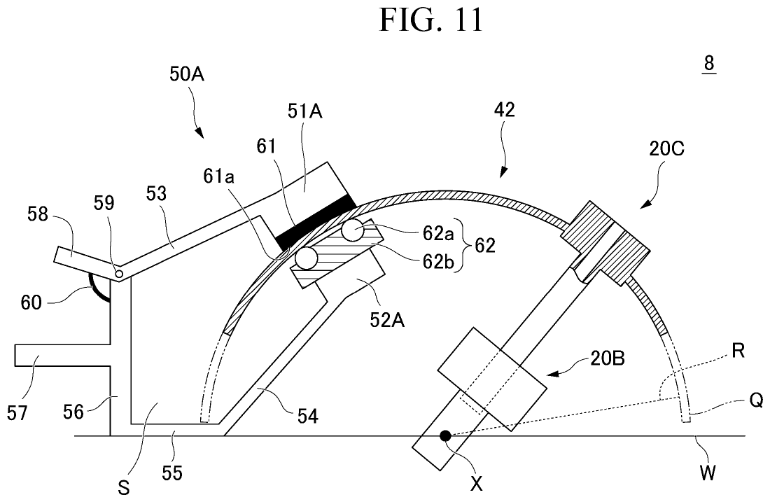

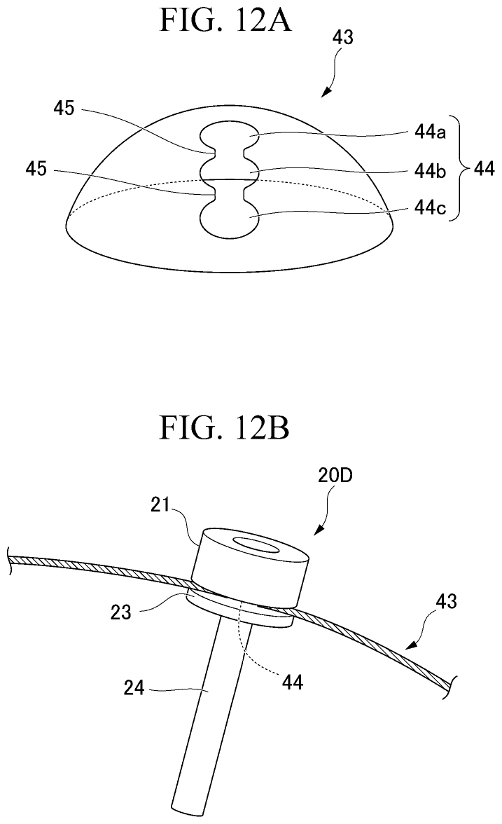

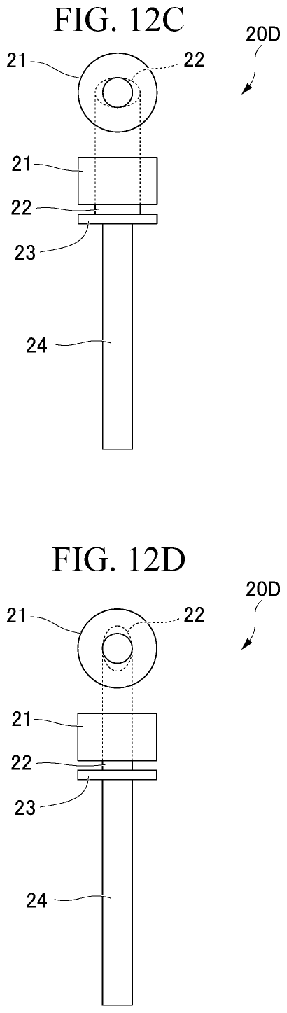

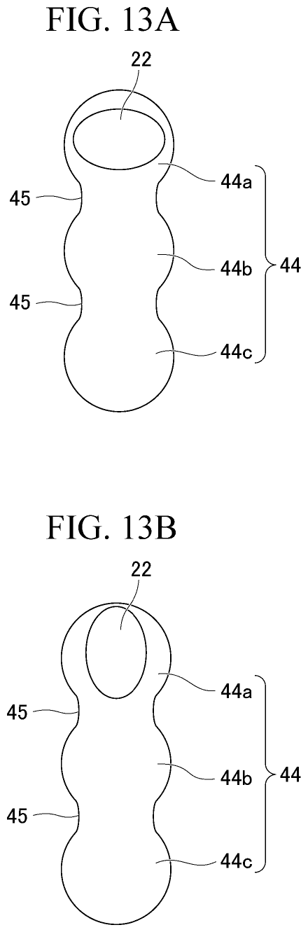

Next, a ninth embodiment of the present invention will be described by referring to FIGS. 12A-12D and 13A-13B.

FIG. 12A is a schematic view showing a configuration of a second retention portion 43 of a medical device retention system according to the present embodiment. A groove portion (groove) 44 is formed in the second retention portion 43. The groove portion 44 is configured from a first hole portion 44a, a second hole portion 44b adjacent to the first hole portion 44a, and a third hole portion 44c adjacent to the second hole portion 44b. As shown in FIG. 12A, the first hole portion 44a communicates to the second hole portion 44b and the second hole portion 44b communicates to the third hole portion 44c such that all of them form the whole groove portion 44. Also, a narrow-width portion 45 is formed in the part at which the first hole portion 44a and the second hole portion 44b communicate with each other. Similarly, the narrow-width portion 45 is formed in the part at which the second hole portion 44b and the third hole portion 44c communicate with each other.

FIG. 12B is a schematic view showing the configuration of the first retention portion 20D of the medical device retention system according to the present embodiment. As shown in FIG. 12B, the first retention portion 20D is inserted into the groove portion 44 of the second retention portion 43, and the first retention portion 20D is supported by the second retention portion 43.

FIGS. 12C and 12D are schematic views showing the configuration of the first retention portion 20D. As shown in FIGS. 12B to 12D, the first retention portion 20D has a mouth portion 21, an engaging portion 22, a fix-ring portion 23, and an elongated portion 24 from the side from which the medical device is inserted. The engaging portion 22 is configured to connect the mouth portion 21 and the fix-ring portion 23, and the engaging portion has a thickness substantially same with that of the second retention portion 43. Also, as shown in FIGS. 12C and 12D, the engaging portion 22 is formed in an elliptic shape to have a major axis and a minor axis. A length of the engaging portion 22 in the major axis direction is longer than a width of the narrow-width portion 45, and a length of the engaging portion 22 in the minor axis direction is shorter than the width of the narrow-width portion 45. The fix-ring portion 23 is formed from a material that is elastically deformable.

An operation example of the first retention portion 20D and the second retention portion 43 having the above-described configurations will be described below.

The user attaches the first retention portion 20D to the second retention portion 43. More specifically, the user inserts the elongated portion 24 of the first retention portion 20D into the first hole portion 44a of the groove portion 44, for example. The user makes the fix-ring portion 23 of the first retention portion 20D to be elastically deformed and go through the first hole portion 44a so as to make the fix-ring portion 23 to abut on the inner circumferential surface side of the second retention portion 43. Accordingly, as shown in FIG. 12B, the second retention portion 43 can be sandwiched by the mouth portion 21 and the fix-ring portion 23 and the first retention portion 20D is supported by the second retention portion 43.

FIGS. 13A and 13B are schematic views showing a relationship between the first retention portion 20D (engaging portion 22) and the second retention portion 43 (groove portion 44).

After the user makes the fix-ring portion 23 of the first retention portion 20D to abut on the inner circumferential surface side of the second retention portion 43, the user rotates the first retention portion 20D relatively to the second retention portion 43 so as to make the major axis direction of the engaging portion 22 to be substantially parallel to the width direction of the narrow-width portion 45 (the state shown in FIG. 13A). In the state shown in FIG. 13A, for example, in a case in which the first retention portion 20D is moving from the first hole portion 44a to the second hole portion 44b, the engaging portion 22 interferes with the narrow-width portion 45. Accordingly, the user can prevent any unintentional movement of the first retention portion 20D by setting the positional relationship of the first retention portion 20D (engaging portion 22) and the second retention portion 43 (groove portion 44) as the state shown in FIG. 13A.

On the other hand, the user can relatively rotate the first retention portion 20D with respect to the second retention portion 43 to change the state in which the major axis direction of the engaging portion 22 is substantially parallel to the width direction of the narrow-width portion 45 (the state shown in FIG. 13A) to the state in which the major axis direction of the engaging portion 22 is substantially vertical to the width direction of the narrow-width portion 45 (the state shown in FIG. 13B). Due to this change, the engaging portion 22 and the narrow-width portion 45 will not interfere with each other. Accordingly, the first retention portion 20D can move from the first hole portion 44a to the second hole portion 44b or the third hole portion 44c. In other words, in the state shown in FIG. 13B, the first retention portion 20D can move along the groove portion 44 while being inserted into the groove portion 44.

Other configuration according to the present embodiment are same with the configurations according to the above-described embodiments. Accordingly, in the present embodiment, it is possible to configure the medical device retention system with a simple configuration, and it is possible to intuitively change the orientation of the held medical device and the like.

According to the present embodiment, the first retention portion 20D can move along the groove portion 44 in the state of being inserted into the groove portion 44. The user can move the first retention portion 20D holding the medical device together with the second retention portion 43 along the virtual spherical surface, and the user can further move the first retention portion 20D relatively to the second retention portion 43 besides. Accordingly, according to the present embodiment, it is possible to further increase the degrees of freedom for changing the orientation of the medical device and the like. Furthermore, according to the present embodiment, in the state in which the medical device is held, the first retention portion 20D can be moved relative to the second retention portion 43 such that a temporary interrupt such as detaching the medical device does not occur.

According to the present embodiment, it is described that the groove portion 44 is configured by the first hole portion 44a, the second hole portion 44b, and the third hole portion 44c, however, the number of the hole portions only has to be equal to 2 or more than 2 and is not limited thereto.

Also, as shown in FIG. 12A, it is described that the groove portion 44 extends in the direction for changing the inclination of the first retention portion 20D inserted thereto, however, the extending direction of the groove portion 44 is not limited thereto and may be any direction. For example, the groove portion 44 may extend along the end portion of the second retention portion 43.

The embodiments of the invention have been described above with reference to the drawings, but specific structures of the invention are not limited to the embodiments and may include various modifications without departing from the scope of the invention.

For example, in the embodiments shown above, the example in which the second retention portion is formed in the partial spherical shape is described, however, a member configured to prevent slipping may be provided at the end portion of the second retention portion. For example, a convex portion projecting in the normal direction with respect to the outer circumferential surface or the inner circumferential surface of the second retention portion may be disposed at the end portion of the second retention portion. In this case, the convex portion engages with the upper-side clipping portion or the lower-side clipping portion of the clamp portion so as to prevent the slipping from the clamp portion of the second retention portion.

In the embodiments shown above, the example of the brake portion configured to fix the movement of the second retention portion is described, however, convex/concave structure may be formed in a range of the brake portion and the second retention portion, wherein the convex/concave structure formed in the range does not give any affect to the movement according to the central point of the second retention portion. In this case, the friction between the brake portion and the second retention portion becomes larger such that the second retention portion can be stably fixed. Also, a convex portion may be formed in the brake portion and a concave portion or a hole for fitting the convex portion may be formed in the second retention portion such that the movement of the second retention portion can be powerfully fixed due to the engagement thereof.

According to the above-described fifth embodiment, the configuration example of using the solenoid and the like to perform the switch operation is described, however, other configurations may be used. For example, the second retention portion may be formed from a material of magnet substance, and the switch from the movement state to the fixing state of the second retention portion may be performed by the brake portion formed from an electromagnet. In this case, it is possible to connect the electromagnet of the brake portion to the power supply portion and connect the power supply portion to the brake operation portion which can perform the switch operation. By turning on/off the brake operation portion, ON/OFF of the magnetic force by the electromagnet of the brake portion can be controlled. For example, during the period when the user presses the switch of the brake operation portion, the electromagnet does not generate the magnet force such that the second retention portion formed from the magnet substance is in the movement state. On the other hand, when the user releases the switch of the brake operation portion, the electromagnet generates the magnetic force, the electromagnet of the brake portion abuts on the second retention portion formed from the magnet substance such that the second retention portion enters the fixing state. Of course, the second retention portion may be in the fixing state during the period when the switch of the brake operation portion is pressed. Also, a surface of the electromagnet of the brake portion formed at the side facing the second retention portion may have a curvature same with the curvature of the second retention portion.

Also, the ball of the guide portion may be formed from the electromagnet to make the guide portion to have the function of the brake portion. In this case, the configuration of the brake portion may be omitted.

Also, in the above-described fifth embodiment, the example of performing wired communication between the driver 67 formed from the solenoid and the like and the brake operation portion 102 via the power supply portion 101 is described, however, a configuration may be realized by providing independent power supply portions therein so as to perform wireless communication. In this case, the power supply portion of the driver 67 may be configured by a small battery and the like which is equipped in the clamp portion 50D.

Also, in the above-described fifth embodiment, the example of disposing the brake operation portion 102 which can perform switch operation in the vicinity of the first retention portion 20 is described, however, the brake operation portion 102 may be formed as a foot switch. In this case, the brake operation portion 102 is not included in the sterilization region during the treatment such that the brake operation portion 102 may be used without performing the sterilization procedures.

Also, in the above-described sixth embodiment, the example of providing the actuator portion 69 in the upper-side clipping portion 51A and the guide portion 62 in the lower-side clipping portion 52A is described, however, the reversed configuration may be provided.

Also, in the above-described ninth embodiment, the example of moving the first retention portion 20D along the groove portion 44 is described, however, the engaging portion 22 and the fix-ring portion 23 of the first retention portion 20D may be provided in the support portion 30. The support portion has such configurations can move along the groove portion.

The embodiments of the invention have been described above with reference to the drawings, but specific structures of the invention are not limited to the embodiments and may include various modifications without departing from the scope of the invention. The invention is not limited to the above-mentioned embodiments and is limited only by the accompanying claims.

What is claimed is:

1. A medical device retention system, comprising:
a tubular first retention portion into which a medical device is inserted, wherein the first retention portion is configured to hold the inserted medical device;
a second retention portion configured to have a spherical shape with a predetermined radius, wherein the first retention portion is supported by the second retention portion, and the second retention portion has a central point at a distal end side of the supported first retention portion; and
a clamp configured to clip the second retention portion,
wherein the clamp is configured to switch the second retention portion between a first state and a second state when the second retention portion is clipped by the clamp,
when the clamp switches the second retention portion into the first state, movement of the second retention portion is fixed, and
when the clamp switches the second retention portion into the second state, the second retention portion is movable along a predetermined surface with the central point as a movement center.

2. The medical device retention system according to claim 1, wherein the clamp has a clipping portion formed with a curvature same with a curvature of the second retention portion.

3. The medical device retention system according to claim 2, wherein the clamp has an upper-side clip formed with the curvature same with the curvature of the second retention portion, and a lower-side clip formed with the curvature same with the curvature of the second retention portion.

4. The medical device retention system according to claim 3, wherein the clamp is configured to switch the second retention portion between the first state and the second state by changing a distance between the upper-side clip and the lower-side clip.

5. The medical device retention system according to claim 4, wherein the upper-side clip is formed from an elastically deformable material.

6. The medical device retention system according to claim 4, further has a biasing member configured to bias the upper-side clip so as to press the upper-side clip toward the second retention portion.

7. The medical device retention system according to claim 6, further has a connection member configured to connect the upper-side clip and the lower-side clip, wherein the biasing member is disposed in the connection member.

8. The medical device retention system according to claim 3, further has a connection member configured to connect the upper-side clip and the lower-side clip,
wherein a space being surrounded by the upper-side clip, the lower-side clip, and the connection member has a volume suitable for at least part of the second retention portion to enter.

9. The medical device retention system according to claim 1, wherein the second retention portion is formed in a partial spherical shape.

10. The medical device retention system according to claim 7, wherein the second retention portion is formed in a partial spherical shape smaller than a hemisphere surface.

11. The medical device retention system according to claim 1, wherein the second retention portion is formed from a transparent material.

12. The medical device retention system according to claim 1, wherein the first retention portion comprises:
a trocar configured to insert the medical device into a body cavity; and
a trocar holder configured to hold the trocar on a wall of the body cavity.

13. The medical device retention system according to claim 1, wherein the first retention portion and the second retention portion are formed integrally.

14. The medical device retention system according to claim 1,
wherein the second retention portion has a groove into which the first retention portion is inserted, and
wherein the first retention portion is configured to be movable along the groove when the first retention portion is inserted into the groove.

15. The medical device retention system according to claim 1, wherein a convex projecting from the second retention portion is be disposed in a vicinity of an end of the second retention portion.

16. The medical device retention system according to claim 1,
wherein the first retention portion is configured to be supported by the second retention portion and movable along a virtual spherical surface while holding the medical device, and
wherein the virtual spherical surface is defined by using the central point of the second retention portion as a center and a predetermined radius.

17. The medical device retention system according to claim 1, wherein the clamp has a guide configured to guide the movement of the second retention portion.

18. The medical device retention system according to claim 1,
wherein the clamp has a brake configured to fix the movement of the second retention portion or release fixation of the movement of the second retention portion, and
wherein a brake operation portion configured to operate the brake is disposed in the vicinity of the first retention portion.

19. The medical device retention system according to claim 18, wherein the brake operation portion is attachable to and detachable from the medical device that is inserted into the first retention portion.
